(19) United States
(12) Patent Application Publication
(10) Pub. No.: US 2025/0270266 A1
(43) Pub. Date: Aug. 28, 2025

(54) METHOD OF MANUFACTURING FIBROIN PROTEIN PRODUCTS

(71) Applicants: Haley Bergman, Aurora, CO (US); Preston Vanderpan, Aurora, CO (US); Avery Williamson, Aurora, CO (US)

(72) Inventors: Haley Bergman, Aurora, CO (US); Preston Vanderpan, Aurora, CO (US); Avery Williamson, Aurora, CO (US)

(21) Appl. No.: 19/061,503

(22) Filed: Feb. 24, 2025

Related U.S. Application Data
(60) Provisional application No. 63/556,595, filed on Feb. 22, 2024.

(51) Int. Cl. C07K 14/435; B01J 19/00; B01J 19/18

(52) U.S. Cl. CPC C07K 14/43504; B01J 19/0013; B01J 19/1862; B01J 2219/00029; B01J 2219/00063; B01J 2219/00162; B01J 2219/00164; B01J 2219/00186

(57) ABSTRACT

A system for producing a fibroin protein product includes a first batch reactor, an inline mixer, a pressurization tapering section, a second batch reactor, and a filtration unit. A method for producing a fibroin protein product includes dissolving fibroin protein in aqueous-based fluid in a first batch reactor, and producing the pretreatment solution and pumping it to an inline mixer. The method includes feeding a salt feed upstream of the inline mixer to mix with the pretreatment solution. The method includes exerting a shear force by the inline mixer to the mixture of the salt feed and the pretreatment solution, producing an intermediate solution, and flowing the intermediate solution to a pressurization tapering section to promote a transformation of the intermediate solution into a fibroin protein product. The method includes filtering the fibroin protein product using a filtration unit to remove unreacted reagents, producing a filtered fibroin protein product.

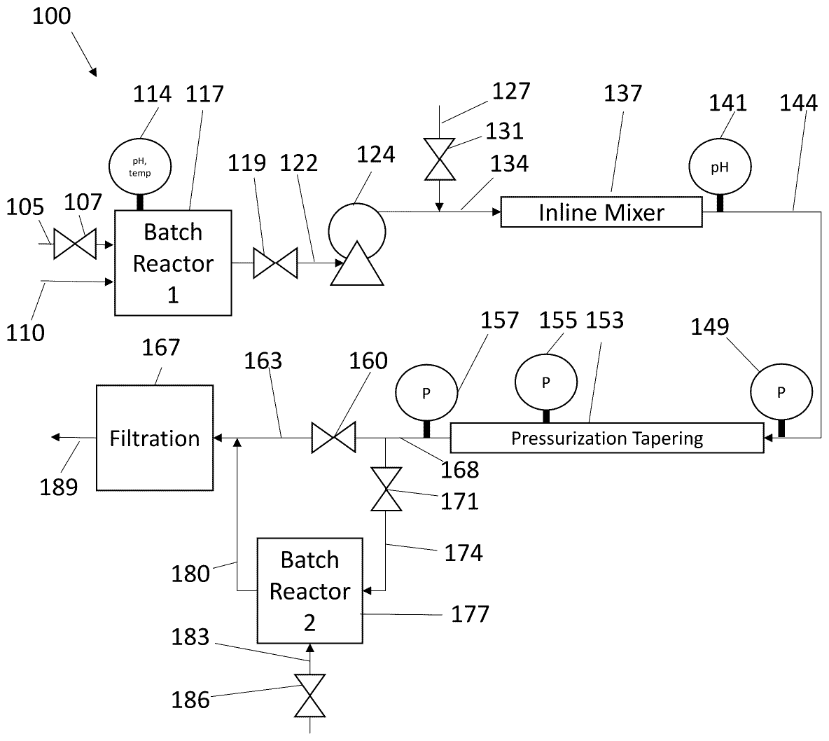

### BACKGROUND

Various synthetic and biologically derived materials exist for use in human tissue construction and repair. In particular, musculoskeletal treatments often rely on grafts of natural or synthetic materials. While synthetic materials, such as hard plastics and glass, are non-toxic and non-immunogenic, they are incapable of supporting necessary cellular activity and often pose a severe risk when integrated with native tissue. On the other hand, natural material grafts often require multiple procedures, incorporate slowly into native tissue, and lose their original mechanical properties. Therefore, the development of inexpensive biomaterials that are capable of supporting cellular and mechanical activity has been a long-standing goal in the biomedical scientific community.

Silk produced from insects and arachnids, such as spiders and silkworms, has recently received attention as a promising biomaterial for use in tissue construction and repair, as it is biocompatible and has immense tensile strength. Unfortunately, manufactured silk typically has poor mechanical properties, undesirable structural features, low reproducibility, and relies on expensive lab equipment and caustic chemical reagents for the artificial spinning systems used for its preparation. The lack of parametric control in the core processes of native silk production restricts the preparation of affordable, high-quality artificial silk on an industrial scale.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a system for producing a fibroin protein product. The system includes a first batch reactor that is configured for receiving a fibroin protein feed and a fluid feed. The fluid feed to the first batch reactor includes a first valve in the fluid feed line. There is a first flow line fluidly connecting the first batch reactor to a first pump with a second valve situated in the first flow line configured to control an effluent from the first batch reactor. There is a second flow line fluidly connecting the first pump to an inline mixer. A salt feed intersects the second flow line upstream of the inline mixer. A third flow line fluidly connects the inline mixer to a pressurization tapering section. A fourth flow line downstream of the pressurization tapering section splits into a fifth flow line, with a fourth valve in it, and a bypass flow line. The fourth valve controls the flow of the effluent from the pressurization tapering section to a second batch reactor. The filtration unit removes unreacted reagents, producing a filtered fibroin protein product, and is downstream from a convergence of the fifth flow line and the bypass flow line.

In another aspect, embodiments disclosed herein relate to a method for producing a fibroin protein product by dissolving a quantity of fibroin protein into an aqueous-based fluid in a first batch reactor, producing a pretreatment solution. The pretreatment solution is pumped to an inline mixer. A salt feed is fed upstream of the inline mixer into the system to mix with the pretreatment solution in the inline mixer and produce a mixture of the salt feed and the pretreatment solution. A shear force is exerted by the inline mixer on the mixture of the salt feed and the pretreatment solution, producing an intermediate solution. The intermediate solution flows to a pressurization tapering section to increase the pressure of the intermediate solution, promoting a transformation of the intermediate solution into a fibroin protein product. The fibroin protein product is filtered using a filtration unit to remove unreacted reagents and produce a filtered fibroin protein product.

Other aspects and advantages of the claimed subject matter will be apparent from the following description and the appended claims.

### BRIEF DESCRIPTION OF DRAWINGS

The FIGURE is a process flow diagram in accordance with one or more embodiments.

### DETAILED DESCRIPTION

In one aspect, embodiments disclosed herein relate to a system for producing a fibroin protein product. In another aspect, embodiments disclosed herein relate to a method for producing a fibroin protein product. Systems and methods described herein involve preparing a pretreatment solution including fibroin protein and then processing the pretreatment solution through various system components to produce a fibroin protein product. Various types of fibroin protein products may be produced using a system and method according to the present disclosure.

In one aspect, embodiments disclosed herein relate to a system and method for producing a pretreatment solution. The pretreatment solution serves the purpose of pretreating fibroin protein prior to further processing to produce fibroin protein products. Disclosed pretreatment solutions may include an aqueous-based fluid, at least one fibroin protein, and one or more salts.

In one or more embodiments, the pretreatment solution includes an aqueous-based fluid. The aqueous-based fluid may include water. The water may be distilled water, deionized water, milli-q water, or combinations thereof. Water may be included in the aqueous-based fluid in an amount ranging from 15 to 85 wt % (weight percent), based on the total weight of the aqueous-based fluid. For example, in one or more embodiments, water may be included in the aqueous-based fluid of a fibroin protein solution in an amount ranging from a lower limit of one of 15, 20, 25, 30, 35, and 40 wt % to an upper limit of one of 60, 65, 70, 75, 80, and 85 wt %, where any lower limit may be paired with any mathematically compatible upper limit.

In one or more embodiments, the aqueous-based fluid may include an alcohol. Suitable alcohols that may be included in an aqueous-based fluid of disclosed compositions include methanol, ethanol, propanol, isopropanol, butanol, isobutanol, and pentanol, among others. In one or more particular embodiments, the aqueous-based fluid includes ethanol.

The aqueous-based fluid may include an alcohol in an amount ranging from 15 to 85 wt %, based on the total weight of the aqueous-based fluid. For example, an alcohol may be included in the aqueous-based fluid in an amount ranging from a lower limit of one of 15, 20, 25, 30, 35, and 40 wt % to an upper limit of one of 60, 65, 70, 75, 80, and 85 wt %, where any lower limit may be paired with any mathematically compatible upper limit.

In one or more embodiments, the aqueous-based fluid includes at least one chloride salt. For example, the aqueous-based fluid may include one or more of sodium chloride, calcium chloride, magnesium chloride, ammonium chloride, among others. In such embodiments, the salt may be included in an amount ranging from 0.05 to 5.0 wt %, based on the total weight of the aqueous-based fluid. In some embodiments, a salt may be added to the aqueous-based fluid in an amount ranging from a lower limit of one of 0.05, 0.1, 0.2, 0.5, and 1.0 wt % to an upper limit of one of 1.0, 2.0, 3.0, 4.0, and 5.0 wt %, where any lower limit may be paired with any mathematically compatible upper limit.

In one or more embodiments, the aqueous-based fluid includes water, an alcohol, and a salt. In one or more particular embodiments, the aqueous-based fluid is Ajisawa's reagent.

In one or more embodiments, the pretreatment solution includes at least one fibroin protein. Fibroin is a protein found in silk produced by various silk-producing genera such as insects and arachnids. The C-terminal and N-terminal domains of fibroin are conserved among all silk-producing genera, allowing for the use of fibroin proteins from various sources in the disclosed compositions. Suitable fibroin proteins may include, but are not limited to, natural proteins produced by spiders such as the Nephila clavipes, and silkworms such as the Bombyx mori; synthetic proteins engineered from yeast, bacteria, and goats; recombinant proteins; and combinations thereof. For example, in one or more embodiments, the fibroin protein included in a fibroin protein solution is a purified spidroin protein, a purified silkworm fibroin protein, a native spider silk web, an engineered fibroin protein from yeast, an engineered protein from bacteria, an engineered protein from goats, or a combination thereof. In other embodiments, fibroin proteins may be recombinant proteins prepared from a one or more natural or synthetic protein, provided that the resulting recombinant protein has the conserved N-terminal and C-terminal domains.

Pretreatment solutions in accordance with the present disclosure may include fibroin protein in an amount ranging from 0.5 to 50% w/v (percent weight by volume). For example, in one or more embodiments, fibroin protein is included in a fibroin protein solution in an amount ranging from a lower limit of one of 0.5, 1.0, 2.0, 5.0, 10 and 15% w/v to an upper limit of one of 20, 25, 30, 35, 40, 45, and 50% w/v, where any lower limit may be paired with any mathematically compatible upper limit.

To pretreat the fibroin protein fed to the process, a quantity of fibroin protein is combined with a quantity of the previously described aqueous-based fluid in a first batch reactor, producing a pretreatment solution. The first batch reactor is equipped with a heating element, a pH monitoring system, a temperature monitoring system, and an agitator. The heating element may be a heating jacket. The temperature monitoring system, in conjunction with the heating element, will maintain a temperature within a range of 50 to 120° C. within the first batch reactor to prevent excess heat that may cause the fibroin proteins to denature. The pH monitoring system will ensure that a pH range is maintained within a range of 4 to 12. In some embodiments, a pH adjustment system may be present to respond to pH readings and adjust accordingly. The contents of the pretreatment solution may be stable in the batch reactor for several hours and up to about 24 hours.]

In some embodiments, prior to the pretreatment process, the silk may be degummed by breaking peptide bonds in the silk, producing the degummed fibroin protein. The degumming process is known by those skilled in the art and can include soaking the fibroin protein in hot water and/or treating it with other materials such as sodium bicarbonate, soap and enzymes. Such a degumming process may be conducted in a tank and the degummed fibroin protein may be fed into the pretreatment process. In other embodiments, the process feed may be already degummed fibroin protein. In these embodiments, a degumming process does not need to occur, however, the fibroin protein may still require a tank for it to be pumped to the next stages of the process. It may also need to be heated or diluted in an aqueous-based fluid in order to achieve a suitable viscosity for further processing.

To feed the aqueous-based fluid to the first batch reactor, a first valve is opened in the fluid feed line. The aqueous-based fluid is first heated in the first batch reactor before the fibroin protein is added. To feed the fibroin protein to the first batch reactor, a valve connected to a degumming or holding tank in a fibroin protein feed line may be opened. In some embodiments, the fibroin protein feed may be suspended in a fluid state to permit flowing the fibroin protein feed through a feed line. In other embodiments, the fibroin protein feed may be in a powdered form. In these embodiments, the fibroin protein feed may be added to the first batch reactor through manual addition or through the use of a charging hopper.

In some embodiments, the aqueous-based fluid is a pre-mixed fluid fed into the first batch reactor. In other embodiments, individual components (e.g., water, alcohol, and salts) may be added separately to the first batch reactor. In such embodiments, the first batch reactor may be equipped with a water feed line, an alcohol feed line, and a solvent feed line for salts in solution. In embodiments in which the salt is added as a powder, it may be added manually, or through the use of a charging hopper.

Once the silk in the pretreatment solution has been suitably degummed and pretreated to form fibroin protein, a second valve is opened to allow the pretreatment solution to be pumped out of the first batch reactor to an inline mixer situated within a flow line. A valve is opened in a salt feed line to feed a salt feed to the flow line upstream of the inline mixer to combine with the pretreatment solution. The salt feed may be in a powdered or a liquid state. The salt feed may contain calcium ions, magnesium ions, or combinations thereof. In one or more embodiments, the salt feed may introduce a chaotropic agent, which may or may not be a salt. In such embodiments, suitable chaotropic agents that may be included include, but are not limited to, Na₂SO₄, (NH₄)₂SO₄CaCl₂, MgCl₂, NaCl, NaBr, NaI, urea, thiourea, guanidine, thiocyanate, guanidinium chloride, guanidinium hydrochloride, and combinations thereof. In particular embodiments, compositions include NaCl.

In one or more embodiments, a chemical chaperone may optionally be added to the inline mixer. A chemical chaperone may be included to stabilize the fibroin protein in the solution and reduce the amount of premature protein folding that may otherwise occur. Suitable chemical chaperones that may be included in fibroin protein solutions include, but are not limited to, urea, glycerol, trehalose, trimethylamine n-oxide, glycine, and combinations thereof. The chemical chaperone may be added via an optional additional feed line as needed.

The inline mixer is a spiral-shaped mixer with a diameter similar to, or less than, the inner diameter of the flow line. When the salt feed and the pretreatment solution center into the inline mixer, they are thoroughly mixed while moving through the flow line. The inline mixer provides a source of shear force to the mixture, producing an intermediate solution. During the process of mixing with shear force in the inline mixer, the fibroin protein begins to transition from an amorphous state to one with a higher crystallinity. Specifically, upon exposure to the shear force, the structure of the fibroin protein in the intermediate solution may be altered such that the β-sheets begin to self-align, providing a structure having stacks of β-sheets. To ensure heat loss does not occur as the mixture moves through the inline mixer, the inline mixer contains a heating element. The heating element may be in the form of heat tracing or a heat jacket. A second pH monitoring system is located downstream of the inline mixer to ensure that a pH range is maintained within a range of 4 to 12, to ensure the mixture remains homogenous throughout the process. In some embodiments, a pH adjustment system may be present to respond to pH readings and adjust accordingly. The pH may be adjusted by adding more of any of the previously described chaotropic agents. Alternatively, a kosmotropic agent, such as NaHCO₃, Na₂CO₃, Cs₂CO₃, NaCl, K₂CO₃KCl, KI, Na₂SO₄, KH₂PO₄, NaH₂PO₄, NaC₆H₇O₇, Na₂C₆H₆O₇, K₃C₆H₅O₇, KBr, (NH₄)₂SO₄, and combinations thereof, may also be used to suitably adjust the pH.

The intermediate solution flows from the inline mixer to a pressurization tapering section to increase the pressure of the intermediate solution, promoting transformation of the intermediate solution into a pressurization tapering section effluent containing the fibroin protein product. The increased pressure and shear forces in the pressurization tapering section extend and partially align the protein chains and induce fibril assembly into β-sheets. The alignment of protein chains and the formation of secondary (or higher order) hierarchical structures results in fiber formation within the viscous solution, resulting in the gelification of the previously viscous protein solution into a hydrogel-like state. The gelification may result from the folding of β-sheets into an axially aligned helical secondary, tertiary or quaternary order structures, resulting in increased mechanical strength of the material. The silk fibers within the solution demonstrate hysteresis (gradual loss of energy under cyclic loading) when mechanically characterized. The viscoelastic properties of this hydrogel-state are highly tunable by altering individual variables of the system including shear forces and pressures based on pipe diameters.

The pressurization tapering section is a segment of piping where the inner diameter decreases in the direction of flow to pressurize the fluid flowing through the segment of piping. For example, the inlet of the pressurization tapering section may have a pipe diameter of 4 inches, while the exit of the pressurization tapering section may have a pipe diameter of 1 inch, thus increasing the pressure of the fluid as it flows through this segment. The pressure of the pressurization tapering section is monitored with a pressure monitoring system situated around and within the section in order to maintain the pressure in a range from 0 to 14,000 psi. For example, the pressure in the pressurization tapering section may have a lower limit of any one of 0, 1, 5, 10, 25, 50, 100, 250, 500, 750, 1,000, 2,000, and 5,000 psi and an upper limit of any one of 10, 100, 1,000, 2,000, 3,000, 4,000, 5,000, 7,500, 10,000, 12,000, and 14,000 psi, where any lower limit may be paired with any mathematically compatible upper limit. The pressure monitoring system may include pressure sensors upstream of the pressurization tapering section, downstream of the pressurization tapering section, and within the pressurization tapering section.

The fibroin protein product may be filtered in a filtration unit after it flows from the pressurization tapering section to yield the filtered fibroin protein product or, in some embodiments, the fibroin protein product may enter a bypass line to be fed to a second batch reactor for further optimization. In embodiments where the fibroin protein product flows directly to the filtration unit, the filtration unit removes unreacted reagents from the fibroin protein product after it exits the pressurization tapering section.

In embodiments where the fibroin protein product is fed to a second batch reactor, the fibroin protein product flows towards the second batch reactor using a valve arrangement to direct the fibroin protein product through a bypass flow line rather than directly to the filtration unit. The second batch reactor has an additive feed line containing a valve. Various additives may be added to further tune the properties of the fibroin protein product. Examples of the additives may be polyethylene glycol, citric acid, or tannic acid. In some embodiments, the second batch reactor may contain an agitator. In some embodiments, the second batch reactor may contain a heating element to ensure the viscosity is low enough to allow the fibroin protein product to move fluidly throughout the system. The valve in the additive feed line is opened to add the additives to the fibroin protein product in the second batch reactor. Following the reaction of the fibroin protein product with an additive, the fibroin protein product flows from the second batch reactor to the filtration unit to remove unreacted reagents and yield the filtered fibroin protein product.

In some embodiments, a control system may be used to monitor and adjust system parameters. The control system may process readings of sensors and instrumentation including, pressure sensors, pH sensors, viscometers, spectroscopy units, and temperature sensors. Based on these values, flow rates and set points of heating elements may be adjusted automatically or manually using a Human Machine Interface (HMI) of a control system.

The FIGURE illustrates the process flow diagram. The first batch reactor 117 receives the fibroin protein feed 110 and the fluid feed 105. The fluid feed 105 contains a first valve 107 to control the addition of fluid to the first batch reactor. The fibroin protein feed 110 may be a fluid moving through a flow line, which may contain a valve, or a powder added to the first batch reactor 117 through a manhole or a charging port. The first batch reactor 117 contains instrumentation 114 that may monitor pH and temperature.

When the fibroin protein has dissolved into the aqueous-based fluid, a second valve 119 is opened to allow the pretreatment solution to flow through a first flow line 122 fluidly connecting the first batch reactor 117 to a first pump 124. The first pump 124 pumps the pretreatment solution through a second flow line 134 that is fluidly connected to an inline mixer 137. A salt feed flows through a flow line 127 to intersect with the second flow line 134 upstream of the inline mixer 137. The valve 131 in the flow line 127 is opened to flow the salt feed into the second flow line 134. The salt feed and the pretreatment solution mix within the inline mixer 137. The inline mixer 137 exerts a shear force to produce an intermediate solution. A pH monitoring system 141 is situated downstream of the inline mixer 137 and upstream of the pressurization tapering section 153.

The intermediate solution flows through a third flow line 144 to the pressurization tapering section 153. The pressure of the intermediate solution increases as it flows through the pressurization tapering section 153. A first pressure sensor 149 is located upstream of the pressurization tapering section 153. A second pressure sensor 155 is located within the pressurization tapering section 153. A third pressure sensor 157 is located downstream of the pressurization tapering section 153.

The effluent 168 of the pressurization tapering section 153, containing the fibroin protein product, splits into two flow lines: a fifth flow line 163 and a bypass flow line 174. The fifth flow line 163 contains a fourth valve 160. When the fourth valve 160 is opened, the fibroin protein product flows to the filtration unit 167 through the fifth flow line 163, producing the filtered fibroin protein product 187. The bypass flow line 174 contains a fifth valve 171. When the fifth valve 171 is opened, the fibroin protein product flows to the second batch reactor 177 through the bypass flow line 174. The second batch reactor 177 also receives an additional feed from an additive feed line 183 containing a valve 186. The valve 186 controls the addition of the additive to the second batch reactor 177. The effluent from the second batch reactor 180 converges with the fifth flow line 163, allowing the effluent from the second batch reactor, containing the fibroin protein product, to flow to the filtration unit 167, producing the filtered fibroin protein product 187.

Embodiments of the present disclosure may provide at least one of the following advantages. The systems and methods described herein use readily available commercial equipment and materials to provide high quality fibroin protein products at large scale. The process can be readily modified to employ additives to tune the final properties of the fibroin protein products.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A system for producing a fibroin protein product, comprising:
a first batch reactor configured for receiving a fibroin protein feed and a fluid feed;
a first flow line fluidly connecting the first batch reactor to a first pump;
a second flow line fluidly connecting the first pump to an inline mixer;
a salt feed intersecting the second flow line upstream of the inline mixer;
a third flow line fluidly connecting the inline mixer to a pressurization tapering section;
a fourth flow line downstream of the pressurization tapering section splitting into a fifth flow line and a bypass flow line;
a fourth valve situated in the fifth flow line configured to control flow of an effluent from the pressurization tapering section to a filtration unit;
a fifth valve situated in the bypass flow line configured to control flow of the effluent from the pressurization tapering section to a second batch reactor; and
the filtration unit downstream from a convergence of the fifth flow line and the bypass flow line configured to remove unreacted reagents, producing a filtered fibroin protein product.

2. The system of claim 1, wherein the first batch reactor comprises a first pH monitoring system.

3. The system of claim 1, wherein the first batch reactor comprises a temperature monitoring system.

4. The system of claim 1, wherein the first batch reactor comprises an agitator.

5. The system of claim 1, wherein the first batch reactor comprises a heating element.

6. The system of claim 1, wherein the inline mixer comprises a heating element.

7. The system of claim 1, further comprising a second pH monitoring system downstream of the inline mixer and upstream of the pressurization tapering section.

8. The system of claim 1, further comprising a pressure monitoring system situated around the pressurization tapering section.

9. The system of claim 8, wherein the pressure monitoring system includes a first pressure sensor upstream of the pressurization tapering section, a second pressure sensor within the pressurization tapering section, and a third pressure sensor downstream of the pressurization tapering section.

10. The system of claim 1, wherein the pressurization tapering section is a segment of piping where an inner diameter decreases to pressurize a fluid flow through the segment of piping.

11. The system of claim 1, wherein the second batch reactor comprises an additive feed line with a valve situated in the additive feed line.

12. A method for producing a fibroin protein product, comprising:
dissolving a quantity of fibroin protein in a quantity of aqueous-based fluid in a first batch reactor, producing a pretreatment solution;
pumping the pretreatment solution to an inline mixer;
feeding a salt feed upstream of the inline mixer to mix with the pretreatment solution in the inline mixer, producing a mixture of the salt feed and the pretreatment solution;
exerting a shear force by the inline mixer to the mixture of the salt feed and the pretreatment solution, producing an intermediate solution;
flowing the intermediate solution to a pressurization tapering section to increase the pressure of the intermediate solution to promote a transformation of the intermediate solution into a fibroin protein product; and
filtering the fibroin protein product using a filtration unit to remove unreacted reagents, producing a filtered fibroin protein product.

13. The method of claim 12, further comprising maintaining a pH within the first batch reactor within a range of 4 to 12.

14. The method of claim 12, further comprising maintaining a temperature within the first batch reactor within a range of 50 to 120° C.

15. The method of claim 12, wherein the salt feed is a liquid.

16. The method of claim 12, wherein the salt feed is a powder.

17. The method of claim 12, wherein the salt feed is selected from the group consisting of calcium salts, magnesium salts, and a combination thereof.

18. The method of claim 12, further comprising maintaining a pH in the intermediate exiting the inline mixer within a range of 4 to 12.

19. The method of claim 12, further comprising maintaining a pressure in the pressurization tapering section within a range of 0 to 14,000 psi.

20. The method of claim 12, further comprising mixing the fibroin protein product with an additive in a second batch reactor prior to filtration.
